(19) United States
(12) Patent Application Publication
(10) Pub. No.: US 2021/0330667 A1
(43) Pub. Date: Oct. 28, 2021

(54) TOPICAL COMPOSITION OF NALBUPHINE AND METHOD FOR TREATING PRURITUS CONDITION, PAIN, OR INFLAMMATORY CONDITION BY USING THE SAME

(71) Applicant: LUMOSA THERAPEUTICS CO., LTD., TAIPEI CITY (TW)

(72) Inventors: David Chih-Kuang CHOU, TAIPEI CITY (TW); Chia-yin LU, TAIPEI CITY (TW)

(21) Appl. No.: 17/368,995

(22) Filed: Jul. 7, 2021

(51) Int. Cl. A61K 31/485; A61K 9/00; A61K 47/10; A61K 47/20; A61K 47/14; A61K 47/32; A61K 47/02; A61K 47/06; A61K 47/26; A61K 47/22; A61K 47/18

(52) U.S. Cl. CPC A61K 31/485; A61K 9/0014; A61K 47/10; A61K 47/20; A61K 47/14; A61K 47/18; A61K 47/02; A61K 47/06; A61K 47/26; A61K 47/22; A61K 47/32

(57) ABSTRACT

Provided is a topical composition of nalbuphine, comprising nalbuphine or a pharmaceutically acceptable salt thereof and at least one pharmaceutically acceptable excipient comprising a penetration enhancer, a thickening agent, a solvent, or any combination thereof. Also provided are a kit comprising a unit dosage of the topical composition of nalbuphine and a method treating pruritus condition, pain, or inflammatory condition by using the topical composition of nalbuphine.

### BACKGROUND OF THE INVENTION

### 1. Field of the Invention

The present invention relates to a topical composition of nalbuphine, a kit comprising a unit dosage of the topical composition of nalbuphine, and a method for treating pruritus condition, pain, or inflammatory condition by using the topical composition of nalbuphine.

### 2. Description of the Prior Arts

Pruritus, or itch, is an uncomfortable skin sensation that provokes a desire to scratch. It may involve the entire skin (generalized pruritus) or only particular areas, such as the scalp, upper back, arms, or groin (localized pruritus). Although itch may be acute, for example, from a bug bite, chronic pruritus originates from many different causes. It is a debilitating condition, comparable to chronic pain, which negatively impacts quality of life. For example, pruritus can cause anger, a feeling of helplessness, and frustration, and the relentless itch can significantly disrupt sleep and concentration. See Blume-Peytavi et al., Atopic dermatitis in children: management of pruritus, J. Eur Acad. Dermatol. Venereol., 26:2-8, 2012; Chang et al., Atopic dermatitis, melatonin, and sleep disturbance., Pediatrics, 134:e397-405, 2014.

Chronic pruritus affects millions of people worldwide, although solid epidemiological data are very limited. Patients with certain diseases and conditions report high incidences of chronic itch, including those with psoriasis, Hodgkin's disease, dialysis patients, and polycythaemica vera. See Metz et al., CME Dermatol., 3:3, 124-143, 2008. Chronic pruritus is also a prevalent symptom in cutaneous T-cell lymphoma, a disease that includes mycosis fungoids and Sezary syndrome. See Meyer et al., Acta Derm, Venereol., 90:12-17, 2010. Pruritus is the most common dermatological complaint in elderly patients. See Beauregard et al., Arch. Dermatol., 123:1638-43, 1987. Itch is often a side effect of certain drugs, such as EGF receptor antagonists. Hu et al., Cutaneous side effects of epidermal growth factor receptor inhibitors: clinical presentation, pathogenesis, and management, J. Am. Acad. Dermatol., 55(2):317-26, 2007.

The chemical name of nalbuphine is 17-(cyclobutylmethyl)-4,5α-epoxymorphinan-3,6α,14-triol, which is a derivative of 14-hydroxymorphine and is structurally related to the opioid μ-receptor agonist oxymorphone and the pioid μ-receptor antagonist, naloxone. Gustein et al. (Chapter 23: Opioid Analgesics, Goodman & Gilman's The Pharmacologic Basis of Therapeutics, 10^{th} Ed., McGraw Hill 2001, pp. 569-619) report that nalbuphine exerts its clinical pharmacologic action by competitively antagonizing the opioid μ-receptor and simultaneously acting as an agonist at the opioid κ-receptor, and thus is a member of the “opioid agonist-antagonist” class of drugs that mechanistically work through this dual pharmacologic process. Nalbuphine has been used to treat acute, chronic, and post-surgical pain. It has been reported that nalbuphine and certain low molecular weight nalbuphine prodrugs with increased lipophilicity (nalbuphine pivalate, nalbuphine enanthate, and nalbuphine dacanoate) can diffuse through skin. See Sung et al., Delivery of nalbuphine and its prodrugs across skin by passive diffusion and iontophoresis, Journal of Controlled Release, 67:1-8, 2000. However, none of the prior arts disclose any topical formulation containing nalbuphine.

Therefore, there is still a need to develop a topical composition of nalbuphine which delivers nalbuphine to the subject in need in a fast and convenient way.

### SUMMARY OF THE INVENTION

In view of the above-mentioned drawbacks of the prior arts, one objective of the present invention is to provide a topical composition of nalbuphine which is faster and more convenient for administration of nalbuphine to subjects in need.

To achieve the foresaid objective, one objective of the present invention is to provide a topical composition of nalbuphine, comprising nalbuphine or a pharmaceutically acceptable salt thereof and at least one pharmaceutically acceptable excipient.

Another objective of the present invention is to provide a method for treating pruritus condition, pain, or inflammatory condition, comprising administering the above-mentioned topical composition of nalbuphine to a subject in need thereof.

Yet another objective of the present invention is to provide a kit comprising a unit dosage of the topical composition of nalbuphine.

In some embodiments, nalbuphine is nalbuphine free base or nalbuphine HCl. In some embodiments, nalbuphine comprised in the topical composition of the present invention may exist in the form of one or more stereoisomers, wherein one or more of those stereoisomers are therapeutically active. In some embodiments, nalbuphine comprised in the topical composition of the present invention is a therapeutically active stereoisomer that is substantially free of other stereoisomers.

The at least one pharmaceutically acceptable excipient comprised in the topical composition of the present invention may be any topically acceptable non-transdermally effective excipient known in the art. In some embodiments, the at least one pharmaceutically acceptable excipient comprises a penetration enhancer, a thickening agent, a solvent, or any combination thereof.

In some embodiments, the penetration enhancer may be any of epidermal penetration enhancers known to those skilled in the art, which include, but are not limited to, alcohols, alkanols, alkanones such as benzyl alcohol, decanol, ethanol, octanol, and propanol; polyols and esters thereof such as butanediol, ethylene glycol, glycerol, 1,2,6 hexanetriol, polyethylene glycol, polyethylene glycol cetyl stearyl 1-octadecanol derivative (such as Promulgen G, which is stearyl alcohol and Cateareth 20 (polyethylene glycol ether of cetearyl alcohol)), propylene glycol monocaprylate, propylene glycol monolaurate and propylene glycol; fatty acids such as lauric acid, oleic acid, and valeric acid; fatty acid esters such as ethyl oleate, isopropyl myristate, and methylpropionate; amides and other nitrogenous compounds such as diethanolamine, dimethylacetamide, dimethylformamide, ethanolamine, 1-methyl-2-pyrrolidone, 2-pyrrolidone, triethanolamine, and urea; diisopropyl adipate, dimethyl isosorbide, ethers such as diethylene glycol monoethyl ether (such as Transcutol P), polyethylene glycol octadecyl ether (such as Brij S20), polyoxyethylene lauryl ether (such as Brij L4 (Brij 30)) and diethylene glycol monomethyl ether; organic acids such as citric acid, salicylic acid, salicylates, and succinic acid; pyrrolidones such as 2-pyrrolidone; 1-substituted azacycloheptan-2-ones, such as 1-n-dodecylcyclazacycloheptan-2-one (laurocapram); sulfoxides such as decylmethylsulfoxide and dimethylsulfoxide, oleyl alcohol, castor oil, 2-n-heptyl-1,3-dioxolane, 2-n-nonyl-1,3-dioxolane, 2-n-undecyl-1,3-dioxolane, 2-n-nonyl-1,3-dioxane, 2-n-undecyl-1,3-dioxane, 2-n-heptylaldehyde-acetal, 2-n-octyl-aldehyde-acetals, e.g., 2-n-octyl-aldehyde-dimethylacetal; 2-n-nonylaldehyde-acetals, 2-n-decylaldehyde-acetals, 3,7-dimethyl-2,6-octadienal (citral) acetals, citronal acetals, 2-n-nonyl-1,3-dioxolane (2-NND), and decanal dimethyl or diethyl acetals; and any combination thereof. In some embodiments, the penetration enhancer comprises dimethyl isosorbide, diethylene glycol monoethyl ether, castor oil, oleyl alcohol, propylene glycol, diisopropyl adipate, ethanol, glycerol, benzyl alcohol, isopropyl myristate, triethanolamine, polyethylene glycol octadecyl ether, polyoxyethylene lauryl ether, polyethylene glycol cetyl stearyl 1-octadecanol derivative, or any combination thereof.

In some embodiments, the thickening agent may be any thickening agents known to those skilled in the art, which include, but are not limited to, microcrystalline cellulose, hydroxypropyl methylcellulose (HPMC), hydroxyethyl cellulose (HEC), methyl cellulose, ethylcellulose, hydroxyethylcellulose, carboxymethylcellulose, hydroxypropyl methylcellulose phthalate, hydroxypropyl cellulose (HPC), modified cellulose derivatives, polyvinylpyrrolidone, Carbopol (carbomer) such as Carbpol 974P, sodium hyaluronate, hyaluronic acid, acacia, dextrin, polyethylene glycol (PEG) 800 to 8000, polysaccharides (such as dextrates, guar gum, acacia, tragacanth and xanthan gum), saturated fatty acids with C12 to C22, polyethers, colloidal silica and methacrylate derivatives, polyethylene oxides, polyoxyethylene-polyoxypropylene copolymers, polyvinyl alcohol, soft paraffin, stearic acid, polyoxyethylene tallow amines such as Ethomeen®. In some embodiments, the thickening agent comprises PEG 4000, soft paraffin, stearic acid, HPC, carbomer, or any combination thereof.

In some embodiments, the solvent may be any solvent known to those skilled in the art, which includes, but is not limited to, alcohol (e.g., methanol, ethanol and higher alcohols), castor oil, 3-[(3-cholamidopropyl) dimethylammonio]-1-propane-sulfonate, cholesterol NF, cholic acid, citric acid, 3-cyclohexene-1-methanol, dehydrated alcohol, deoxycholic acid, diethylene glycol monoethyl ether, diisopropanolamine (1:9), a4-dimethyl-a-(4-methyl-3-pentenyl), ethoxydiglycol, ethoxylated alcohol, ethyl alcohol, ethylene glycol, fatty alcohol citrate, glycerin, 1-hexadecanol, 1,2,6-hexanetriol, hexylene glycol, hydroxypropyl betacyclodextrin, isopropyl alcohol, isopropyl myristate, isopropyl palmitate, lecithin, mineral oil, 2-methyl-1,3-propanediol, oleyl alcohol, phosphoric acid, polyethylene glycols, polyethylene glycol (PEG) 200 to 600, polyethylene glycol 1000 monocetyl ether, polyethylene glycol monostearate, polyoxyl 20 cetostearyl ether, polyoxypropylene 15-stearyl ether, polysorbates, polysorbate 20/40/60/80, sodium hydroxide, potassium hydroxide, propylene carbonate, propylene glycol, propylethylene glycol 4, neopentyl alcohol, SD alcohol 40, sodium lauryl sulfate, sorbitan monostearate, sorbitan stearate, taurodeoxycholic acid, triacetin, triethylene glycol, trimethylene glycol, dimethicone, petrolatum, propylene glycol dicaprylate/dicaprate, vegetable oils, phenoxyethanol, water, tetrahydrofuran, aromatics (e.g., benzene and toluene), alkanes (e.g., pentane, hexane and heptane), ketones (e.g., nd methyl ethyl ketone), chlorinated hydrocarbons (e.g., chloroform, carbon tetrachloride, methylene chloride and ethylene dichloride), dimethyl sulfoxide (DMSO), caprylic/capric triglyceride (medium chain triglycerides, such as Miglyol 810), liquid paraffin, cetyl alcohol, cetostearyl alcohol, stearyl alcohol, carboxylpolymethylene, polycarbophil, polyethylene glycol, sorbitan esters, magnesium lauryl sulfate, a wax such as white wax, glycerol stearate, PEG-100 stearate, sorbitol, polypropylene glycol, levulinic acid, lauryl lactate, polyoxyethylene 20 sorbitan monooleate, aluminum starch octenylsuccinate, propylene glycol stearate or acetates (e.g., ethyl acetate). In some embodiments, the solvent comprises PEG 400, diisopropyl adipate, benzyl benzoate, N-methyl-2-pyrrolidone (NMP), isopropyl myristate (IPM), caprylic/capric triglyceride, liquid paraffin, DMSO, sodium lauryl sulfate, cetyl alcohol, stearyl alcohol, carboxylpolymethylene, white wax, sodium hydroxide, glycerol stearate, PEG-100 stearate, sorbitol, polypropylene glycol, levulinic acid, lauryl lactate, polyoxyethylene 20 sorbitan monooleate, aluminum starch octenylsuccinate, propylene glycol stearate, hexylene glycol, water, or any combination thereof.

In some embodiments, the at least one pharmaceutically acceptable excipient further comprises a preservative, an antioxidant, or any combination thereof.

In some embodiments, the preservative may include, but is not limited to, antimicrobial preservatives such as methyl paraben (nipagin), propyl paraben (nipasol), butylparaben, ethylparaben, methylparaben, propyl paraben potassium, and propyl paraben sodium. In some embodiments, the preservative comprises methyl paraben, propyl paraben, or a combination thereof.

In some embodiments, the antioxidant may include, but is not limited to, butylated hydroxytoluene (BHT), butylated hydroxyanisole (BHA), ethylenediaminetetraacetic acid (EDTA), propyl gallate, ascorbic acid, citric acid, ascorbyl palmitate, alpha-tocopherol, and alpha-tocopherol acetate.

In some embodiments, the topical composition may be in a form of gel, ointment, lotion, foam, or cream.

In some embodiments, the topical composition of the present invention comprises an effective amount of nalbuphine. As used herein, an “effective amount” is an amount effective for treating the pruritus condition, pain, or inflammatory condition. In some embodiments, the topical composition of the present invention comprises nalbuphine in an amount of 0.1 wt % to 4 wt %, 0.5 wt % to 3 wt %, 1 wt % to 2 wt %.

In some embodiments, the topical composition of the present invention is in a form of cream. In some embodiments, the topical composition in a form of cream comprises nalbuphine in an amount of 0.1 wt % to 4 wt %, and at least one pharmaceutically acceptable excipient in an amount of 96% to 99.9 wt %. In one embodiment, the topical composition of the present invention in a form of cream comprises nalbuphine HCl in an amount of 0.1 wt % to 4 wt %; cetyl alcohol in an amount of 3.5 wt %; stearyl alcohol in an amount of 3.5 wt %; sodium lauryl sulfate in an amount of 1 wt %; methyl paraben in an amount of 0.1 wt %; propyl paraben in an amount of 0.05 wt %; carboxylpolymethylene in an amount of 0.2 wt %; propylene glycol in an amount of 5 wt %; sodium hydroxide in an amount of 0.03 wt %; soft paraffin in an amount of 12.50 wt %; liquid paraffin in an amount of 6.50 wt %; and water in an amount sufficient to provide a total weight of 100% of the topical composition.

In one embodiment, the topical composition of the present invention in a form of cream comprises nalbuphine HCl in an amount of 0.1 wt % to 4 wt %; glycerol in an amount of 12 wt %; DMSO in an amount of 40 wt %; propylene glycol in an amount of 20 wt %; ethanol in an amount of 3 wt %; and water in an amount sufficient to provide a total weight of 100% of the topical composition.

In one embodiment, the topical composition of the present invention in a form of cream comprises nalbuphine free base in an amount of 0.1 wt % to 4 wt %; soft paraffin in an amount of 61 wt %; white wax in an amount of 5 wt %; propylene glycol stearate in an amount of 8 wt %; polyethylene glycol cetyl stearyl 1-octadecanol derivative in an amount of 7 wt %; aluminum starch octenylsuccinate in an amount of 5 wt %; hexylene glycol in an amount of 3 wt %; and water in an amount sufficient to provide a total weight of 100% of the topical composition.

In some embodiments, the topical composition of the present invention is in a form of ointment. In some embodiments, the topical composition in a form of ointment comprises nalbuphine in an amount of 0.1 wt % to 4 wt %, and at least one pharmaceutically acceptable excipient in an amount of 96% to 99.9 wt %.

In one embodiment, the topical composition of the present invention in a form of ointment comprises nalbuphine HCl in an amount of 0.1 wt % to 4 wt %; polyoxyethylene 20 sorbitan monooleate in an amount of 4 wt %; methyl paraben in an amount of 0.20 wt %; propyl paraben in an amount of 0.05 wt %; soft paraffin in an amount of 10 wt %; and liquid paraffin in an amount sufficient to provide a total weight of 100% of the topical composition.

In one embodiment, the topical composition of the present invention in a form of ointment comprises nalbuphine free base in an amount of 2%; castor oil in an amount of 14 wt %; isopropyl myristate in an amount of 18 wt %; caprylic/capric triglyceride in an amount of 10 wt %; diisopropyl adipate in an amount of 15 wt %; soft paraffin in an amount of 36 wt %; and stearic acid in an amount of 5 wt %.

In one embodiment, the topical composition of the present invention in a form of ointment comprises nalbuphine free base in an amount of 4 wt %; isopropyl myristate in an amount of 5 wt %; oleyl alcohol in an amount of 10 wt %; caprylic/capric triglyceride in an amount of 10 wt %; liquid paraffin in an amount of 25 wt %; diisopropyl adipate in an amount of 10 wt %; soft paraffin in an amount of 30 wt %; stearic acid in an amount of 6 wt %.

In some embodiments, the topical composition of the present invention is in a form of gel. In some embodiments, the topical composition in a form of gel comprises nalbuphine in an amount of 0.1 wt % to 4 wt %, and at least one pharmaceutically acceptable excipient in an amount of 96% to 99.9 wt %.

In one embodiment, the topical composition of the present invention in a form of gel comprises nalbuphine HCl in an amount of 0.1 wt % to 4 wt %; carbomer in an amount of 2 wt %; sodium hydroxide in an amount of 1.25 wt %; and water in an amount sufficient to provide a total weight of 100% of the topical composition.

In one embodiment, the topical composition of the present invention in a form of gel comprises nalbuphine HCl or nalbuphine free base in an amount of 0.1 wt % to 4 wt %; ethanol in an amount of 20 wt %; DMSO in an amount of 14.5 wt %; propylene glycol in an amount of 7.5 wt %; glycerol in an amount of 5 wt %; benzyl alcohol in an amount of 2 wt %; polyethylene glycol octadecyl ether in an amount of 3 wt %; polyoxyethylene lauryl ether in an amount of 3 wt %; and water in an amount sufficient to provide a total weight of 100% of the topical composition.

In one embodiment, the topical composition of the present invention in a form of gel comprises nalbuphine HCl or nalbuphine free base in an amount of 0.1 wt % to 4 wt %; ethanol in an amount of 20 wt %; DMSO in an amount of 28 wt %; dimethyl isosorbide in an amount of 5.5 wt %; propylene glycol in an amount of 10 wt %; lauryl lactate in an amount of 5 wt %; polyoxyethylene lauryl ether in an amount of 4 wt %; and water in an amount sufficient to provide a total weight of 100% of the topical composition.

In one embodiment, the topical composition of the present invention comprises nalbuphine HCl in an amount of 0.1 wt % to 4 wt %; ethanol in an amount of 20 wt %; levulinic acid in an amount of 2.5 wt %; DMSO in an amount of 20 wt %; dimethyl isosorbide in an amount of 15 wt %; propylene glycol in an amount of 20 wt %; lauryl lactate in an amount of 4 wt %; and water in an amount sufficient to provide a total weight of 100% of the topical composition.

In one embodiment, the topical composition of the present invention comprises nalbuphine HCl in an amount of 3 wt %; polypropylene glycol in an amount of 82 wt %; diethylene glycol monoethyl ether in an amount of 15 wt %.

In one embodiment, the topical composition of the present invention comprises nalbuphine free base in an amount of 2 wt %; dimethyl isosorbide in an amount of 15 wt %; PEG 400 in an amount of 40 wt %; diethylene glycol monoethyl ether in an amount of 10 wt %; diisopropyl adipate in an amount of 18 wt %; and PEG 4000 in an amount of 15 wt %.

In one embodiment, the topical composition of the present invention comprises nalbuphine HCl in an amount of 4 wt %; glycerol stearate in an amount of 4 wt %; cetyl alcohol in an amount of 3 wt %; PEG-100 stearate in an amount of 2.5 wt %; soft paraffin in an amount of 8 wt %; isopropyl myristate in an amount of 4.5 wt %; sorbitol in an amount of 3.8 wt %; benzyl alcohol in an amount of 1 wt %; and water in an amount sufficient to provide a total weight of 100% of the topical composition.

In one embodiment, the topical composition of the present invention comprises nalbuphine free base in an amount of 0.1 wt % to 4 wt %; carbomer in an amount of 2 wt %; PEG 400 in an amount of 20 wt %; propylene glycol in an amount of 10 wt %; triethanolamine in an amount of 0.75 wt %; isopropyl myristate in an amount of 4 wt %; and water in an amount sufficient to provide a total weight of 100% of the topical composition.

The term“pruritus” as used herein, is defined as a sensation driving the urge to scratch, and may be acute or chronic. In some embodiments, the pruritus condition can be uremic pruritus, atopic dermatitis, prurigo, nervous dermatitis, aquagenic pruritus, photosensitive dermatosis, idiopathic pruritus, Lichen simplex chronicus, prurigo nodularis, psoriasis, cholestatic pruritus, contact dermatitis, seborrheic dermatitis, autosensitization dermatitis, caterpillar dermatitis, eczema, asteatosis, senile pruritus cutaneous, insect sting, scabies, urticaria, herpes, impetigo, tinea, lichen, acne vulgaris, or visceral diseases complicated with pruritus, brachioradial pruritus, burn-induced pruritus, cancer-induced pruritus, neuropathic pruritus, morphine-induced pruritus, multiple sclerosis-associated pruritus, post-herpetic pruritus, bullous pemphigoid, Netherton syndrome, or itching from poison or any other stimulus. In some embodiments, the pruritus condition is uremic pruritus, atopic dermatitis, or prurigo nodularis.

In some embodiments, the topical composition of the present invention may be administered to the subject in need at an appropriate interval, such as administered daily, every other day, three times a week, twice a week, once a week, or at others. In some embodiments, the composition of the present invention is administered until the skin condition in the affected area is completely healed.

In some embodiments, the nalbuphine is administered to the subject in need in an amount effective to treat the pruritus condition, pain, or inflammatory condition. In some embodiments, the dose of nalbuphine depends upon the form of the composition of nalbuphine, the subject to be treated, the age, body weight and/or other characteristics of the subject to be treated, the preference and experience of the attending physician, and/or the nature of the skin condition to be treated. The dose of nalbuphine administered may be the same with or different from the doses disclosed herein. In some embodiments, the dose may be chosen by physicians or those skilled in the arts in accordance with the present invention.

Specifically, the nalbuphine may be administered to the subject in need in a daily dose of up to about 0.1 mg/day to about 100 mg/day on the skin. In some embodiments, the nalbuphine is administered to the subject in need in a daily dose of about 5 mg/day to about 50 mg/day on the skin. The total daily dose may be delivered once per day, or divided between multiple doses. In some embodiments, the nalbuphine is administered 1, 2, 3, 4, or 5 times per day.

In some embodiments, the topical composition of nalbuphine of the present invention may be administered before, after, or concurrently with any traditional treatment of pruritus condition, pain, or inflammatory condition, such as chlorpheniramine maleate, calamine lotion, Ichderm and zinc oxide (ZnO).

In some embodiments, the topical composition of the present invention may be provided as a kit comprising the unit dosage of the topical composition within a container, wherein the container may be sterile. In some embodiments, the kit further comprises a label or printed instructions instructing the use of the topical composition of nalbuphine of the present invention to treat the pruritus condition, pain, or inflammatory condition.

Other objectives, advantages and novel features of the invention will become more apparent from the following detailed description.

### DETAILED DESCRIPTION OF THE PREFERRED EMBODIMENTS

The following embodiments are provided to demonstrate the present invention.

Cream Formulations

Formulation C1

The Formulation C1 is formulated in accordance with the following Table 1 to obtain a cream. Water is in an amount sufficient to provide a total weight of 100% of the Formulation C1.

TABLE 1

| | Amount |
|---|---|
| Ingredient | (wt % of the composition) |
| Nalbuphine HCl | 0.1 to 4 |
| Cetyl alcohol | 3.5 |
| Stearyl alcohol | 3.5 |
| Sodium lauryl sulfate | 1 |
| Methyl paraben | 0.1 |
| Propyl paraben | 0.05 |
| Carboxylpolymethylene | 0.2 |
| Propylene glycol | 5 |
| Sodium hydroxide | 0.03 |
| Soft paraffin | 12.50 |
| Liquid paraffin | 6.50 |
| Water | q.s. |

Formulation C2

The Formulation C2 is formulated in accordance with the following Table 2 to obtain a cream. Water is in an amount sufficient to provide a total weight of 100% of the Formulation C2.

TABLE 2

| | Amount |
|---|---|
| Ingredient | (wt % of the composition) |
| Nalbuphine HCl | 0.1 to 4 |
| Glycerol | 12 |
| DMSO | 40 |
| Propylene glycol | 20 |
| Ethanol | 3 |
| Water | q.s. |

Formulation C3

The Formulation C3 is formulated in accordance with the following Table 3 to obtain a cream. Water is in an amount sufficient to provide a total weight of 100% of the Formulation C3.

TABLE 3

| | Amount |
|---|---|
| Ingredient | (wt % of the composition) |
| Nalbuphine free base | 0.1 to 4 |
| Soft paraffin | 61 |
| White wax | 5 |
| Propylene glycol stearate | 8 |
| Polyethylene glycol cetyl stearyl 1- | 7 |
| octadecanol derivative | |
| Aluminum starch octenylsuccinate | 5 |
| Hexylene glycol | 3 |
| Water | q.s. |

Ointment Formulations

Formulation O1

The Formulation O1 is formulated in accordance with the following Table 4 to obtain an ointment. Liquid paraffin is in an amount sufficient to provide a total weight of 100% of the Formulation O1.

TABLE 4

| | Amount |
|---|---|
| Ingredient | (wt % of the composition) |
| Nalbuphine HCl | 0.1 to 4 |
| Polyoxyethylene 20 sorbitan monooleate | 4 |
| Methyl paraben | 0.20 |
| Propyl paraben | 0.05 |
| Soft paraffin | 10 |
| Liquid paraffin | q.s. |

Formulation O2

The Formulation O2 is formulated in accordance with the following Table 5 to obtain an ointment.

TABLE 5

| | Amount |
|---|---|
| Ingredient | (wt % of the composition) |
| Nalbuphine free base | 2 |
| Castor Oil | 14 |
| IPM | 18 |
| Caprylic/capric triglyceride | 10 |
| Diisopropyl adipate | 15 |
| Soft paraffin | 36 |
| Stearic Acid | 5 |

Formulation O3

The Formulation O3 is formulated in accordance with the following Table 6 to obtain an ointment.

TABLE 6

| | Amount |
|---|---|
| Ingredient | (wt % of the composition) |
| Nalbuphine free base | 4 |
| IPM | 5 |
| Oleyl Alcohol | 10 |
| Caprylic/capric triglyceride | 10 |
| Liquid Paraffin | 25 |
| Diisopropyl adipate | 10 |
| Soft paraffin | 30 |
| Stearic Acid | 6 |

Gel Formulations

Formulation G1

The Formulation G1 is formulated in accordance with the following Table 7 to obtain a gel. Water is in an amount sufficient to provide a total weight of 100% of the Formulation G1.

TABLE 7

| | Amount |
|---|---|
| Ingredient | (wt % of the composition) |
| Nalbuphine HCl | 0.1 to 4 |
| Carbomer | 2 |
| Sodium hydroxide | 1.25 |
| Water | q.s. |

Formulation G2

The Formulation G2 is formulated in accordance with the following Table 8 to obtain a gel. Water is in an amount sufficient to provide a total weight of 100% of the Formulation G2.

TABLE 8

| | Amount |
|---|---|
| Ingredient | (wt % of the composition) |
| Nalbuphine HCl or | 0.1 to 4 |
| Nalbuphine free base | |
| Ethanol | 20 |
| DMSO | 14.5 |
| Propylene glycol | 7.5 |
| Glycerol | 5 |
| Benzyl alcohol | 2 |
| Polyethylene glycol octadecyl ether | 3 |
| Polyoxyethylene lauryl ether | 3 |
| Water | q.s. |

Formulation G3

The Formulation G3 is formulated in accordance with the following Table 9 to obtain a gel. Water is in an amount sufficient to provide a total weight of 100% of the Formulation G3.

TABLE 9

| | Amount |
|---|---|
| Ingredient | (wt % of the composition) |
| Nalbuphine HCl or | 0.1 to 4 |
| Nalbuphine free base | |
| Ethanol | 20 |
| DMSO | 28 |
| Dimethyl isosorbide | 5.5 |
| Propylene glycol | 10 |
| Lauryl lactate | 5 |
| Polyoxyethylene lauryl ether | 4 |
| Water | q.s. |

Formulation G4

The Formulation G4 is formulated in accordance with the following Table 10 to obtain a gel. Water is in an amount sufficient to provide a total weight of 100% of the Formulation G4.

TABLE 10

| | Amount |
|---|---|
| Ingredient | (wt % of the composition) |
| Nalbuphine HCl | 0.1 to 4 |
| Ethanol | 20 |
| Levulinic acid | 2.5 |
| DMSO | 20 |
| Dimethyl isosorbide | 15 |
| Propylene glycol | 20 |
| Lauryl lactate | 4 |
| Water | q.s. |

Formulation G5

The Formulation G5 is formulated in accordance with the following Table 11 to obtain a gel.

TABLE 11

| | Amount |
|---|---|
| Ingredient | (wt % of the composition) |
| Nalbuphine HCl | 3 |
| Polypropylene glycol | 82 |
| Diethylene glycol monoethyl ether | 15 |

Formulation G6

The Formulation G6 is formulated in accordance with the following Table 12 to obtain a gel.

TABLE 12

| | Amount |
|---|---|
| Ingredient | (wt % of the composition) |
| Nalbuphine free base | 2 |
| Dimethyl isosorbide | 15 |
| PEG 400 | 40 |
| Diethylene glycol monoethyl ether | 10 |
| Diisopropyl adipate | 18 |
| PEG 4000 | 15 |

Formulation G7

The Formulation G7 is formulated in accordance with the following Table 13 to obtain a gel. Water is in an amount sufficient to provide a total weight of 100% of the Formulation G7.

TABLE 13

| | Amount |
|---|---|
| Ingredient | (wt % of the composition) |
| Nalbuphine HCl | 4 |
| Glycerol stearate | 4 |
| Cetyl alcohol | 3 |
| PEG-100 stearate | 2.5 |
| Soft paraffin | 8 |
| Isopropyl myristate | 4.5 |
| Sorbitol | 3.8 |
| Benzyl alcohol | 1 |
| Water | q.s. |

Formulation G8

The Formulation G8 is formulated in accordance with the following Table 14 to obtain a gel. Water is in an amount sufficient to provide a total weight of 100% of the Formulation G8.

TABLE 14

| | Amount |
|---|---|
| Ingredient | (wt % of the composition) |
| Nalbuphine free base | 0.1 to 4 |
| Carbomer | 2 |
| PEG 400 | 20 |
| Propylene glycol | 10 |
| Triethanolamine | 0.75 |
| Isopropyl myristate | 4 |
| Water | q.s. |

Even though numerous characteristics and advantages of the present invention have been set forth in the foregoing description, together with details of the features of the invention, the disclosure is illustrative only. Changes may be made in the details within the principles of the invention to the full extent indicated by the broad general meaning of the terms in which the appended claims are expressed.

What is claimed is:

1. A topical composition of nalbuphine, comprising nalbuphine or a pharmaceutically acceptable salt thereof and at least one pharmaceutically acceptable excipient.

2. The topical composition as claimed in claim 1, wherein the nalbuphine is nalbuphine free base or nalbuphine HCl.

3. The topical composition as claimed in claim 1, wherein the at least one pharmaceutically acceptable excipient comprises a penetration enhancer, a thickening agent, a solvent, or any combination thereof.

4. The topical composition as claimed in claim 1, wherein the topical composition is in a form of gel, ointment, lotion, foam, or cream.

5. The topical composition as claimed in claim 1, comprising nalbuphine HCl in an amount of 0.1 wt % to 4 wt %; cetyl alcohol in an amount of 3.5 wt %; stearyl alcohol in an amount of 3.5 wt %; sodium lauryl sulfate in an amount of 1 wt %; methyl paraben in an amount of 0.1 wt %; propyl paraben in an amount of 0.05 wt %; carboxylpolymethylene in an amount of 0.2 wt %; propylene glycol in an amount of 5 wt %; sodium hydroxide in an amount of 0.03 wt %; soft paraffin in an amount of 12.50 wt %; liquid paraffin in an amount of 6.50 wt %; and water in an amount sufficient to provide a total weight of 100% of the topical composition.

6. The topical composition as claimed in claim 1, comprising nalbuphine HCl in an amount of 0.1 wt % to 4 wt %; glycerol in an amount of 12 wt %; DMSO in an amount of 40 wt %; propylene glycol in an amount of 20 wt %; ethanol in an amount of 3 wt %; and water in an amount sufficient to provide a total weight of 100% of the topical composition.

7. The topical composition as claimed in claim 1, comprising nalbuphine free base in an amount of 0.1 wt % to 4 wt %; soft paraffin in an amount of 61 wt %; white wax in an amount of 5 wt %; propylene glycol stearate in an amount of 8 wt %; polyethylene glycol cetyl stearyl 1-octadecanol derivative in an amount of 7 wt %; aluminum starch octenylsuccinate in an amount of 5 wt %; hexylene glycol in an amount of 3 wt %; and water in an amount sufficient to provide a total weight of 100% of the topical composition

8. The topical composition as claimed in claim 1, comprising nalbuphine HCl in an amount of 0.1 wt % to 4 wt %; polyoxyethylene 20 sorbitan monooleate in an amount of 4 wt %; methyl paraben in an amount of 0.20 wt %; propyl paraben in an amount of 0.05 wt %; soft paraffin in an amount of 10 wt %; and liquid paraffin in an amount sufficient to provide a total weight of 100% of the topical composition.

9. The topical composition as claimed in claim 1, comprising nalbuphine free base in an amount of 2%; castor oil in an amount of 14 wt %; isopropyl myristate in an amount of 18 wt %; caprylic/capric triglyceride in an amount of 10 wt %; diisopropyl adipate in an amount of 15 wt %; soft paraffin in an amount of 36 wt %; and stearic acid in an amount of 5 wt %.

10. The topical composition as claimed in claim 1, comprising nalbuphine free base in an amount of 4 wt %; isopropyl myristate in an amount of 5 wt %; oleyl alcohol in an amount of 10 wt %; caprylic/capric triglyceride in an amount of 10 wt %; liquid paraffin in an amount of 25 wt %; diisopropyl adipate in an amount of 10 wt %; soft paraffin in an amount of 30 wt %; stearic acid in an amount of 6 wt %.

11. The topical composition as claimed in claim 1, comprising nalbuphine HCl in an amount of 0.1 wt % to 4 wt %; carbomer in an amount of 2 wt %; sodium hydroxide in an amount of 1.25 wt %; and water in an amount sufficient to provide a total weight of 100% of the topical composition.

12. The topical composition as claimed in claim 1, comprising nalbuphine HCl or nalbuphine free base in an amount of 0.1 wt % to 4 wt %; ethanol in an amount of 20 wt %; DMSO in an amount of 14.5 wt %; propylene glycol in an amount of 7.5 wt %; glycerol in an amount of 5 wt %; benzyl alcohol in an amount of 2 wt %; polyethylene glycol octadecyl ether in an amount of 3 wt %; polyoxyethylene lauryl ether in an amount of 3 wt %; and water in an amount sufficient to provide a total weight of 100% of the topical composition.

13. The topical composition as claimed in claim 1, comprising nalbuphine HCl or nalbuphine free base in an amount of 0.1 wt % to 4 wt %; ethanol in an amount of 20 wt %; DMSO in an amount of 28 wt %; dimethyl isosorbide in an amount of 5.5 wt %; propylene glycol in an amount of 10 wt %; lauryl lactate in an amount of 5 wt %; polyoxyethylene lauryl ether in an amount of 4 wt %; and water in an amount sufficient to provide a total weight of 100% of the topical composition.

14. The topical composition as claimed in claim 1, comprising nalbuphine HCl in an amount of 0.1 wt % to 4 wt %; ethanol in an amount of 20 wt %; levulinic acid in an amount of 2.5 wt %; DMSO in an amount of 20 wt %; dimethyl isosorbide in an amount of 15 wt %; propylene glycol in an amount of 20 wt %; lauryl lactate in an amount of 4 wt %; and water in an amount sufficient to provide a total weight of 100% of the topical composition.

15. The topical composition as claimed in claim 1, comprising nalbuphine HCl in an amount of 3 wt %; polypropylene glycol in an amount of 82 wt %; diethylene glycol monoethyl ether in an amount of 15 wt %.

16. The topical composition as claimed in claim 1, comprising nalbuphine free base in an amount of 2 wt %; dimethyl isosorbide in an amount of 15 wt %; PEG 400 in an amount of 40 wt %; diethylene glycol monoethyl ether in an amount of 10 wt %; diisopropyl adipate in an amount of 18 wt %; and PEG 4000 in an amount of 15 wt %.

17. The topical composition as claimed in claim 1, comprising nalbuphine HCl in an amount of 4 wt %; glycerol stearate in an amount of 4 wt %; cetyl alcohol in an amount of 3 wt %; PEG-100 stearate in an amount of 2.5 wt %; soft paraffin in an amount of 8 wt %; isopropyl myristate in an amount of 4.5 wt %; sorbitol in an amount of 3.8 wt %; benzyl alcohol in an amount of 1 wt %; and water in an amount sufficient to provide a total weight of 100% of the topical composition.

18. The topical composition as claimed in claim 1, comprising nalbuphine free base in an amount of 0.1 wt % to 4 wt %; carbomer in an amount of 2 wt %; PEG 400 in an amount of 20 wt %; propylene glycol in an amount of 10 wt %; triethanolamine in an amount of 0.75 wt %; isopropyl myristate in an amount of 4 wt %; and water in an amount sufficient to provide a total weight of 100% of the topical composition.

19. A method for treating pruritus condition, pain, or inflammatory condition, comprising administering the topical composition as claimed in claim 1 to a subject in need thereof.

20. The method as claimed in claim 19, wherein the pruritus condition is uremic pruritus, atopic dermatitis, or prurigo nodularis.
